(19) United States
(12) Patent Application Publication
(10) Pub. No.: US 2006/0009797 A1
(43) Pub. Date: Jan. 12, 2006

(54) Anoscope

(76) Inventor: David N. Armstrong, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/224,470

(22) Filed: Sep. 12, 2005

Related U.S. Application Data
(63) Continuation-in-part of application No. 10/042,998, filed on Apr. 12, 2002.
(60) Provisional application No. 60/260,724, filed on Jan. 9, 2001.

(51) Int. Cl. A61M 29/00

(52) U.S. Cl. 606/197

(57) ABSTRACT

An anoscope for ligating internal hemorrhoids has an outer tubular body with lateral hemorrhoid-receiving apertures coinciding with the normal location of internal hemorrhoids in man, and a closed-ended, generally bullet-shaped head to close the distal end of the anoscope and to protect the operator from escaping gas and bowel content. A rotatable inner obturator has lateral apertures that register with the lateral apertures of the outer tubular body when the anoscope is open and intermediate slats that occlude the lateral apertures when the anoscope is closed.

### CROSS REFERENCE TO RELATED APPLICATIONS

This application is a continuation-in-part of co-pending U.S. patent application Ser. No. 10/042,998, filed Jan. 9, 2002, which claims priority to Provisional Patent Application Ser. No. 60/260,724, filed Jan. 9, 2001.

### TECHNICAL FIELD

This invention concerns an anoscope that is used during surgical procedures for removal or other treatment of hemorrhoids of man. More particularly, the anoscope includes hemorrhoid-receiving apertures that are positioned circumferentially about a tubular body that are in the proper anatomical positions to simultaneously present the hemorrhoids.

### BACKGROUND OF THE INVENTION

Hemorrhoids in man may be located within the anal canal (internal hemorrhoids) or external to the anal canal (external hemorrhoids). Internal hemorrhoids are located proximal to the dentate line, which is a circumferential arrangement of anal glands located approximately 2-3 cm within the anal canal.

Because internal hemorrhoids are located proximal to the dentate line, where somatic sensory nerves are absent, they can be treated using non-operative procedures such as ligation, injection, infra-red coagulation or other means of destroying the hemorrhoidal tissue. This procedure avoids a surgical hemorrhoidectomy which is much more uncomfortable for the patient and is associated more potential complications.

Alternative methods of treating internal hemorrhoids also includes suture ligation, stapling, cryo-ablation, infra-red coagulation, injection sclerotherapy, or radiofrequency ablation, each of which may be performed in conjunction with the anoscope described herein.

Internal hemorrhoids in man are located in the left lateral, right anterior and right posterior locations. With the patient in the supine position, this translates into the 3, 7 and 11 o'clock locations from the operator's perspective. With the patient in the prone position, this translates into the 9, one and five o'clock position. With the patient in the left lateral position (for instance immediately following colonoscopy), this translates into the 6, 11 and 1 o'clock positions.

My U.S. patent application Ser. No. 10/042,998 discloses a system of ligating internal hemorrhoids using a configured cylindrical anoscope, with lateral apertures, which correspond to the normal anatomic location of internal hemorrhoids in man. The current invention describes improvements upon the basic system and anoscope, which makes the procedure of hemorrhoidal ablation safer and easier for the operator, and therefore less uncomfortable for the patient.

### SUMMARY OF THE INVENTION

The anoscope is to be used for eradicating internal anal hemorrhoids in man. It includes a tubular body and an obturator rotatively received in the tubular body. Either the tubular body or the obturator has at its distal end a bullet-shaped head that closes the distal end of the anoscope.

Three lateral hemorrhoid-receiving apertures are located in the tubular body at the normal anatomic locations of the internal anal hemorrhoids in man. Anal hemorrhoids are located in the left lateral, right anterior and right posterior locations, or 3, 7 and 11 o'clock positions when man is in the supine position. The three hemorrhoid-receiving apertures formed in the tubular body of the anoscope each extend approximately one sixth of the circumference of the tubular body and are equally circumferentially spaced from on another.

The bullet-shaped head that forms the closed distal end configuration of the anoscope strengthens the anoscope and reduces the likelihood of damage of the anoscope “straps” or “fins” that form the apertures of the anoscope. The closed distal end of the anoscope also tends to prevent explosive release of gas and stool through the anal canal of the patient that might occur when using an open-ended anoscope.

In one embodiment of the invention a rotary obturator is used in the tubular body of the anoscope that includes an inner cylinder also with apertures at 3, 7 and 11 o'clock positions that extend approximately one sixth of the circumference of the cylinder. The obturator is telescopically received in and is rotatable within the tubular body. The obturator may be rotated so that its apertures are aligned with the hemorrhoid-receiving apertures of the tubular body and the hemorrhoids will distend through the aligned apertures and are presented for ablation or other treatment by the physician. Also, the obturator may be rotated to move its apertures out of alignment with the hemorrhoid-receiving apertures of the tubular body to block the presentation of the hemorrhoids.

In another embodiment of the invention, the rotary obturator has only one aperture for selective alignment with one of the hemorrhoid-receiving apertures. This allows the presentation of the hemorrhoids one at a time.

The anoscope may include an alignment feature that indicates the relative rotary positions of the tubular body and the obturator.

The bullet-shaped head of the anoscope may be mounted on the distal end of the tubular body or on the distal end of the obturator. The head closes the otherwise open distal end of the anoscope.

In another embodiment of the invention, the obturator is telescopically received in the outer tubular body of the anoscope but is not intended to rotate. It carries the bullet-shaped head that functions to close the otherwise open distal end of the outer tubular body. The obturator may be removed from the outer tubular body when the anoscope is properly inserted in the anal canal to allow the physician to have more room to perform the surgical procedures.

A modified multiple hemorrhoidal ligator is available for use with the anoscope that includes a head assembly that is angulated at 1-90 degrees in order to more effectively ligate internal hemorrhoids. Another multiple ligator includes a rotatory ligator shaft assembly, whereby the shaft of the hemorrhoidal ligator may be rotated to predetermined positions, coinciding with the normal hemorrhoidal positions in man (for instance 3, 7 and 11 o'clock). The shaft of the ligator may be rotated by means of lateral extensions extending from the proximal aspect of the shaft of the ligator, which may be rotated by the operator by applying rotational force, using the thumb or index finger. The combination of the modified closed-ended anoscope, the rotatory obturator, the angulated ligator head, the rotatory ligator and a means to rotate the ligator renders the procedure of hemorrhoidal ligation safer and easier to perform. The anoscope described herein contributes to the safety and ease of manipulation of the ligator.

### BRIEF DESCRIPTION OF THE DRAWINGS

FIG. 1 is a perspective view of the anoscope, showing the obturator withdrawn from the tubular body.

FIG. 2 is a side view of the anoscope showing the obturator withdrawn from the tubular body.

FIG. 3 is a cross-sectional view of the assembled anoscope, showing the hemorrhoid-receiving apertures of the tubular body and the apertures of the obturator in alignment with each other.

FIG. 4 is a cross-sectional view of the anoscope, similar to FIG. 3, but illustrating a modified obturator that has one aperture that registers with the hemorrhoid-receiving apertures of the tubular body one at a time.

FIG. 5 is a side cross-sectional view of the proximal end of the tubular body of the anoscope, showing how a removable handle is connectable thereto.

FIG. 6 is an end view of the proximal end of the tubular body of the anoscope, showing the removable handle, and showing an alternate connector position for the removable handle.

FIG. 7 is a side view of another embodiment of the anoscope, with the bullet shaped head of the anoscope mounted to the obturator.

FIG. 8 is a side view of another embodiment of the anoscope, with a modified obturator.

### DETAILED DESCRIPTION

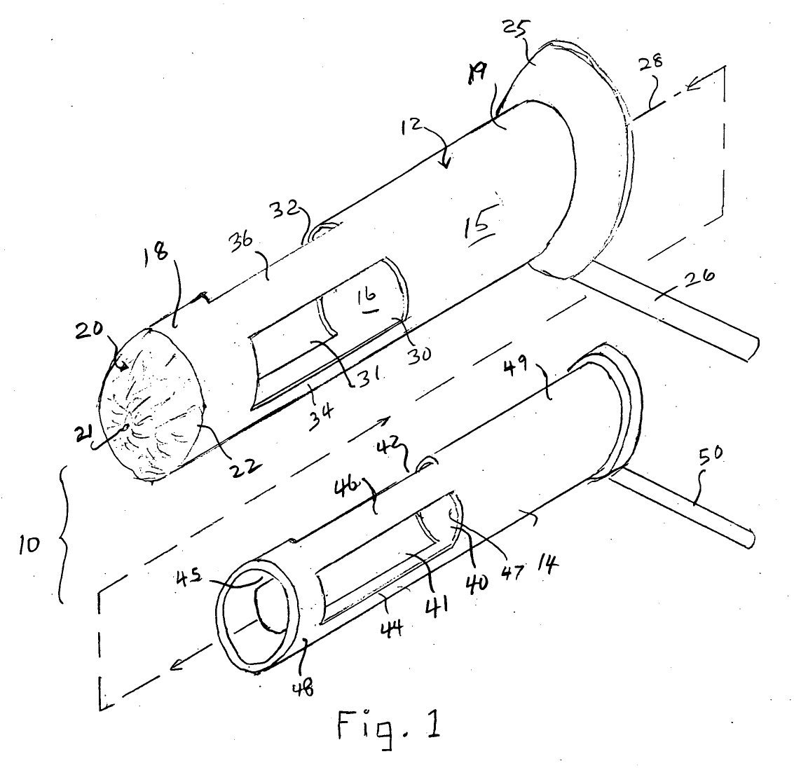

Referring now in more detail to the drawings, in which like numerals indicate like parts throughout the several views, FIG. 1 shows an anoscope 10, an external tubular body 12 and an internal tubular obturator 14. The tubular body 12 includes a substantially cylindrically-shaped side wall 15 that defines an interior 16, a distal end 18 for insertion in the anal canal of a patient and a proximal end 19 for positioning at the entrance of the anal canal.

In this embodiment, a bullet-shaped head 20 is mounted on the distal end 18 of the tubular body. The bullet-shaped head includes a rounded end 21 extending away from the tubular body 12 and a circular end 22 that is mounted to and merges into the cylindrically shaped side wall of the tubular body 12.

A frustum-shaped flange 25 is mounted about the proximal open end 19 of the tubular body 12. A handle 26 extends from the flange in a sloped, radial direction from the longitudinal axis 28 of the tubular body 12.

Hemorrhoid-receiving apertures 30-32 are formed in the cylindrically shaped side wall 15 of the external tubular body 12. The apertures 30-32 are elongated, with their lengths extending parallel to the longitudinal axis 28 of the tubular body 12. The apertures 30-32 are defined by the intermediate support straps 34, 35 and 36. Each hemorrhoid-receiving aperture 30-32 has a width that extends circumferentially about the cylindrically shaped side wall 25 of the tubular body 12, with each aperture extending circumferentially approximately 60°. The intermediate support straps 34-36 preferably extend circumferentially 60° about the cylindrically shaped side wall.

As best shown in FIG. 3, the hemorrhoid-receiving apertures 30-32 are located at the 3 o'clock, 7 o'clock, and 11 o'clock positions about the tubular body 12. This corresponds to the correct anatomical positions of the anal hemorrhoids of man in the supine position.

The hemorrhoid-receiving apertures 30-32 are located at the distal end 18 of the tubular body 12, extending from the distal end toward an intermediate position between the distal end and the proximal end 19 of the tubular body. This leaves an unapertured portion of the tubular body at its proximal end 19.

When the tubular body 12 is inserted into the anal canal, the hemorrhoid-receiving apertures 30-32 will be located in the vicinity of the hemorrhoids of man, but the proximal end 19 that has no apertures will be located at the entrance to the anal canal.

Obturator 14 is cylindrically shaped and is sized and shaped so as to be telescopically received within and rotatable within the interior 16 of the tubular body 12. It forms an interior space 47. The obturator includes elongated apertures 41 and 42 formed therethrough that are sized and shaped to simultaneously align with the apertures of the tubular body. The elongated apertures 40-42 each also extend 60° about the circumference of the obturator, leaving intermediate support straps 44, 45 and 46. The support straps also extend 60° about the circumference of the obturator.

The apertures 40-42 of the obturator are also located adjacent the distal end 48 of the obturator, leaving an unapertured proximal end 49. The apertures 40-42 are shaped, sized, and positioned so as to accurately register with the hemorrhoid-receiving apertures 30-32 of the tubular body 12.

Handle 50 is mounted to the proximal end 49 of the obturator, and extends at an angle from the longitudinal axis of the obturator that is similar to the angle at which the handle 26 extends from the longitudinal axis of the tubular body 12. With this arrangement, when the handles 26 and 50 are oriented adjacent and parallel to each other, the elongated apertures 40-42 of the obturator will be in complete registration with the hemorrhoid-receiving apertures 30-32 of the tubular body 12.

When the three elongated apertures 40-42 of obturator 14 are in registration with the hemorrhoid-receiving apertures 30-32 of the tubular body 12, and when the handles 26 and 50 are oriented in the 12 o'clock position with the patient in the supine position, the apertures 30-32 and 40-42 will be at the 3, 7 and 11 o'clock positions as shown in FIG. 3, which will be in registration with the natural positions of the hemorrhoids of the human body. The hemorrhoids tend to protrude through and be presented within the interior 16 of the anoscope and within the interior 47 of the obturator, and can be viewed by the physician. With this arrangement, the handles become an alignment means for indicating the rotary position of the hemorrhoid-receiving apertures of the outer tubular body with respect to hemorrhoids of the patient and of the rotary position of the apertures of the obturator with respect to the outer tubular body.

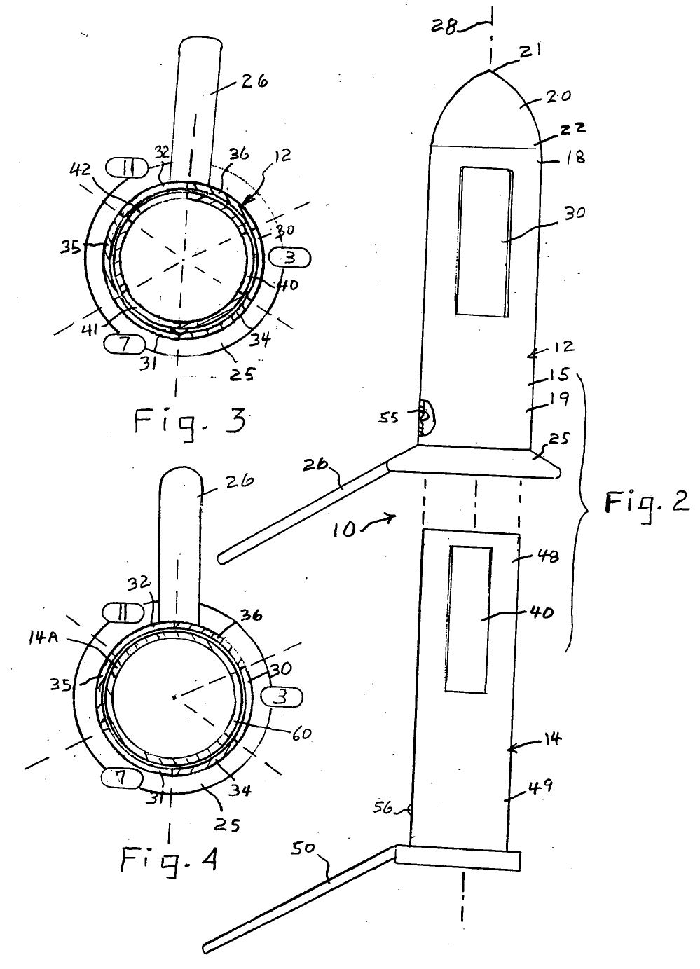

Another alignment means may include a recess 55 (FIG. 2) on the inside surface of the tubular body 12 at the interior proximal end 19, and a protrusion 56 at the exterior proximal end 49 of the obturator. The alignment protrusion 56 registers with the alignment recess 55 when the obturator 14 is moved telescopically into the interior 16 of the tubular body 12 and the apertures are aligned. The alignment protrusion 56 tends to “click” into the alignment recess 55, making a slight clicking noise, and tending to resiliently maintain the obturator in its position where its elongated apertures 40-42 are maintained in alignment with the hemorrhoid-receiving apertures 30-32.

FIG. 4 illustrates a modified obturator 14A that includes only one elongated aperture 60 that registers with any one of the hemorrhoid-receiving apertures 30-32. Since there is only one elongated aperture 60 formed in the obturator 14A, the obturator is capable of registering with the hemorrhoid-receiving apertures one at a time, by rotating the obturator.

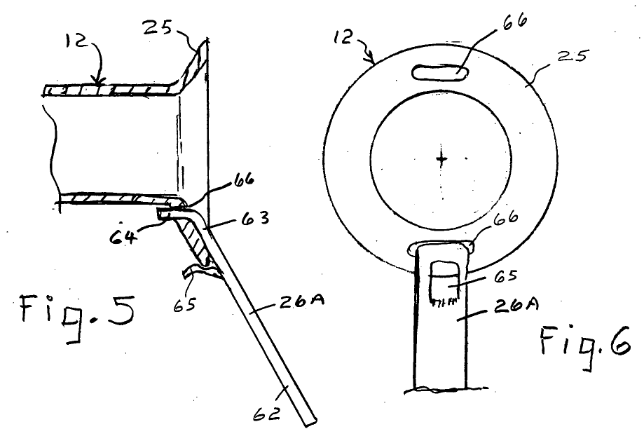

While the handles of the tubular body 12 and the obturator 14 have been indicated as being permanently mounted, it is possible to mount the handles 26 and 50 in adjustable positions. For example, FIGS. 5 and 6 illustrate a handle 26A that is releasably connected to the frustum shaped flange 25 of the external tubular body 12 of the anoscope. An elongated flat bar 62 that forms a handle has a connector end 63 formed with an angled terminal end 64 and a tang 65 that is struck from the bar at a position removed from the angled terminal end. The frustum shaped flange 25 has a connector opening 66 formed therein and the angled terminal end 64 is inserted through the connector opening 66. The tang 65 engages the outer perimeter of the frustum shaped flange 25 so that the handle becomes rigidly, but releasably, mounted to the tubular body 12.

As shown in FIG. 6, more than one connector opening 66 can be formed in the frustum shaped flange 25 so that the handle 26A can be connected at more than one position about the frustum shaped flange. Since the connector openings 66 will be located in predetermined positions about the frustum shaped flange 25, the positions of the hemorrhoid-receiving apertures 30-32 will be understood by the position of the handle.

If desired, the removable handle may be used by the physician to insert and orient the anoscope and then removed from the anoscope. Also, the handles 26 and 50 may be of different dimensions and shapes to be compatible with their uses.

In the embodiment shown in FIGS. 1 and 2, the bullet-shaped head 20 closes the distal end of the obturator 12. When in use, the head 20 engages the adjacent surfaces of the anal canal so that internal gasses and matter are retarded passing from the bowels and through the anoscope. The sealing of the obturator at its distal end from the passage of gas or fecal matter into the interior 16 of the tubular body and the interior 47 of the obturator protects the physician. Also, when the anoscope is in its proper position within the anal canal of the human body with its apertures 30-32 and 40-42 aligned with the hemorrhoids of the patient, the hemorrhoids can be washed or otherwise cleansed or treated with the head 20 maintaining the distal end of the anoscope sealed from the internal aspect of the bowels.

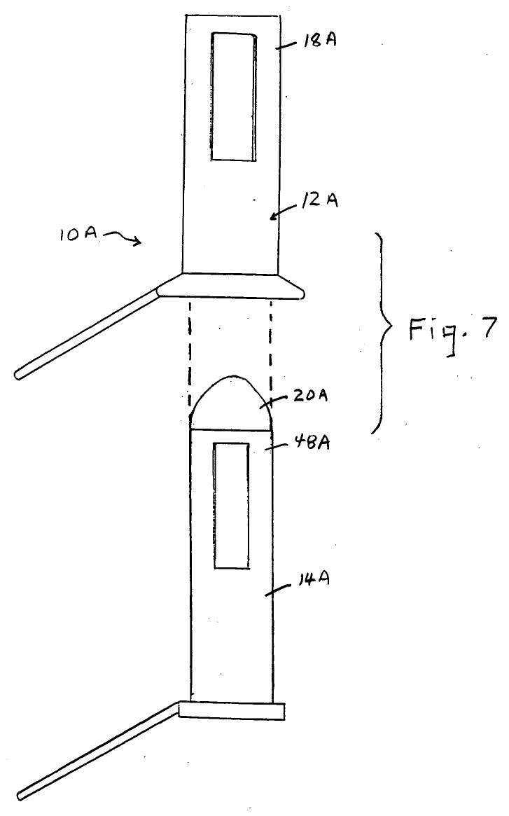

FIG. 7 illustrates a modified anoscope 10A, whereby the bullet-shaped head 20A is mounted to the distal end 48A of the obturator 14A, and the tubular body 12A is open-ended. With this arrangement, when the obturator 14A is fully telescopically received in the tubular body 12A, the bullet shaped head 20A will protrude from the open end of the tubular body 12A, thereby closing the distal end 18A of the tubular body. With this arrangement, the obturator 14A can be withdrawn from the tubular body 18A for access by the physician to the portions of the anal canal that extend beyond the tubular body 12A.

It will be noted that the anoscope will be used with its apertures always aligned with the natural positions of the hemorrhoids of man. In the preferred embodiment, the handles 26 and 50 of the tubular body and the obturator will always be oriented toward the spine of the patient to achieve proper rotary position of the apertures 30-32 and 40-42.

Alternative embodiments of the anoscope may be constructed that include locating the apertures and handle at other relative positions, in order to facilitate hemorrhoidal ligation, with the patient in alternative positions. For example, with the patient in prone position, and the handle at the 12 or 6 o'clock position, the apertures are then located at 9, 1 and 5 o'clock positions, from the operators perspective. Alternatively, with the patient lying in the left lateral position, and the handle of the anoscope parallel to the patients spine (i.e. 9 or 3 o'clock position), the apertures are then located at the 6, 11 and 1 o'clock positions. This latter scenario occurs during colonoscopy, when the patient lays in the left lateral position (i.e. lying on their left side). This is an ideal time to perform hemorrhoidal ligation, since the patient is already sedated, has undergone a mechanical bowel prep, and is under hemodynamic monitoring. The alternative positions of the handle and apertures therefore makes combined colonoscopy and hemorrhoidal ligation an efficient and simple procedure.

The relative locations of the anoscope handle, and the lateral apertures is therefore variable, depending on the position of the patient, and the preference of the operating surgeon. To facilitate the procedure, and to make all potential combinations of handle and apertures available to the operator, an alternative embodiment of the anoscope incorporates a handle, which rotates around the axis of the anoscope. In this embodiment, all potential combinations of handle and apertures are available, and the handle can be “preset” to any desired position. For example the handle may be set and the 12 o'clock position and the apertures at 3, 7 and 11 o'clock, for a patient in supine position. Alternatively, the handle may be preset at 9 o'clock and the apertures at 6, 11 and 1 o'clock for a patient in the left lateral position. This embodiment of the anoscope is therefore more convenient and versatile for the procedure of hemorrhoidal ligation and therefore makes the procedure easier for the operator and less uncomfortable for the patient.

The open-ended configuration of the anoscope has the limitation of allowing gas and bowel content to escape, once the obturator is removed. This is not only very unpleasant for the operator, but potentially dangerous. The current invention describes a closed-ended, generally bullet-shaped anoscope, which reduces the likelihood of this happening and protects the operator. In addition the closed-ended configuration adds mechanical stability to the intervening straps, located between the apertures. The mechanical support afforded by the distal closed-ended tip prevents fracture of the straps, which could potentially cause injury to the patient

When the inner cylinder is rotated 60 degrees, into the “open” position, the apertures of the inner and outer cylinders now register, and the internal hemorrhoids protrude into the interior of the anoscope, ready for ligation. The closed end of the anoscope reduces the likelihood of escape of any material through the anoscope.

Internal hemorrhoids may protrude to various degrees into the interior of the anoscope, depending on their size. It is difficult to ligate the smaller hemorrhoids if they protrude very little in to the lumen of the anoscope. When ligating with a suction ligator such as described by Ahmed (U.S. Pat. No. 6,149,659), the distal tip of the suction ligator may be angulated in order for the suction tip of the ligator to effectively make end-on contact with a hemorrhoid, so facilitating ligation. The angulation may be fixed from 180-90 degrees, more preferably from 120-60 degrees or most preferably at 45 degrees, to the axis of the ligator shaft. In an alternative embodiment, the angulation of the ligator tip may be variable, and adjusted by the operator depending on the individual anatomy of the patient.

Prior art also requires the ligator to be reinserted three separate times, at a different angle in order to ligate each of the three individual internal hemorrhoids. Rather than re-inserting the suction ligator multiple times at different angles, the shaft of the suction ligator may be rotated about its own axis, so the angulated head makes end-on contact with each hemorrhoid, without removing it from the anoscope.

The shaft of the ligator may be rotated by means of rotating the shaft for instance by utilizing lateral extensions extending from the proximal aspect of the shaft of the ligator. These lateral extensions may be rotated by the operator by applying rotational force, using the thumb or index finger. The ligator shaft may be rotated to pre-designated points, to register with the lateral apertures of the anoscope i.e. at 3, 7 and 11 o'clock, or 9, 1 and 0.5 o'clock and so forth. Arrival at the pre-designated point may be indicated by visual, auditory or tactile means. In one embodiment, the operator may feel a “click” as the shaft of the ligator and the anoscope apertures register. This may be easily accomplished by having grooves and protuberances on the shaft of the ligator and its housing at the appropriate locations. Other embodiments of the alignment means may include a system of colors, figures or numbers arrayed circumferentially on the proximal aspect of the ligator shaft and its housing.

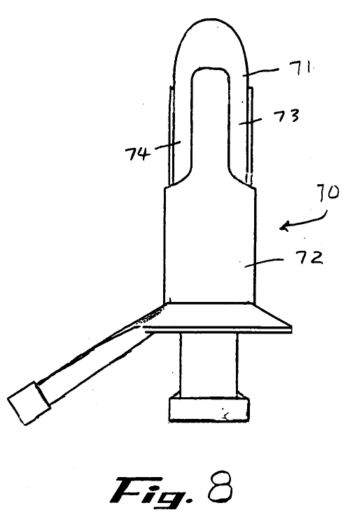

FIG. 8 shows another embodiment of the anoscope 70 that includes an obturator 71 that has a bullet-shaped head that is telescopically received in and protrudes from the distal end of the outer tubular body 72. The outer tubular body 72 has its hemorrhoid-receiving apertures 73, 74 etc. positioned as described above, at the 3, 7 and 11 o'clock positions of the patient. The obturator 71 does not have the circumferentially spaced apertures as described above and is not intended to be rotated, but is for selectively closing the open distal end of the outer tubular body and for ease of insertion in the anal canal. Once the anoscope is properly positioned in the anal canal, the obturator 71 may be removed from the tubular body 72 out through its proximal end.

Although preferred embodiments of the invention have been disclosed in detail herein, it will be obvious to those skilled in the art that variations and modifications of the disclosed embodiments can be made without departing from the spirit and scope of the invention as set forth in the following claims.

What is claimed is:

1. An anoscope for insertion in the anal canal of a human comprising
an elongated tubular body having a longitudinal axis and a substantially cylindrically-shaped side wall that defines an interior, the tubular body having a distal end for insertion in the anal canal and a proximal end for positioning at the entrance of the anal canal,
the cylindrically shaped side wall defining three elongated hemorrhoid-receiving apertures extending through and along the length of the cylindrically-shaped side wall,
the elongated hemorrhoid-receiving apertures extending from adjacent the distal end to an intermediate position between the distal end and the proximal end of the tubular body leaving a non-apertured portion of the tubular body adjacent the proximal end of the tubular body,
the hemorrhoid-receiving apertures located at positions about the tubular body at the normal anatomic locations of the hemorrhoids in the anal canal for simultaneously presenting hemorrhoids in the interior of the tubular body without repositioning the anoscope and permitting simultaneous access through the hemorrhoid-receiving apertures to the normal locations for internal hemorrhoids of the anal canal, and positioning the non-apertured portion of the tubular body to the entrance to the anal canal, and
a handle mounted to the proximal end of the tubular body at a predetermined position with respect to the apertures such that by orienting the handle with respect to the patient all of the hemorrhoid-receiving apertures are simultaneously oriented at the anatomical positions of hemorrhoids of the patient.

2. The anoscope of claim 1, and further including:
an obturator positioned within the tubular body, said obturator having a cylindrical side wall with elongated apertures formed there through that are sized and shaped to simultaneously align with the apertures of the tubular body,
such that the obturator can be rotated to have its apertures simultaneously register with the hemorrhoid-receiving apertures of the tubular body or rotated to simultaneously close the hemorrhoid-receiving apertures.

3. The anoscope of claim 2, wherein said obturator includes a handle that aligns with the handle of the tubular body when the apertures of the obturator are aligned with the hemorrhoid-receiving apertures of the tubular body.

4. The anoscope of claim 1 and further including:
an obturator positioned within and rotatable with respect to the tubular body,
said obturator having a cylindrical side wall with an elongated aperture formed there through that is sized and shaped to align with any one of the apertures of said tubular body,
alignment means for indicating the rotary position of the aperture of the obturator with respect to the tubular body,
such that the obturator can be rotated with respect to the tubular body to have its aperture register one at a time with selected ones of the hemorrhoid-receiving apertures of the tubular body or to close all of the hemorrhoid-receiving apertures of the tubular body.

5. The anoscope of claim 4, wherein the alignment means comprises a handle mounted to the obturator.

6. The anoscope of claim 5, wherein said handle of the obturator is connectable to said obturator at more than one location about the obturator.

7. The anoscope of claim 4, wherein the alignment means comprises a recess and a protrusion that registers with the recess.

8. The anoscope of claim 1, wherein
the hemorrhoid-receiving apertures of the cylindrically shaped side wall of the tubular body each have a width approximately equivalent to one-sixth of the circumference of the cylindrically shaped side wall of the tubular body, and
the hemorrhoid-receiving apertures being spaced apart about the cylindrical wall to be at the three o'clock, seven o'clock and eleven o'clock positions about the cylindrical wall when the anoscope is inserted in the anal canal with the handle oriented in the twelve o'clock position.

9. The anoscope of claim 1, and further including a head mounted on and closing the distal end of said tubular body.

10. The anoscope of claim 2, and further including a head mounted on and closing the distal end of said obturator.

11. The anoscope of claim 2 and further including alignment means for indicating the rotary position of the apertures of the obturator with respect to the anoscope.

12. An anoscope comprising:
a tubular body including a cylindrically shaped side wall, a distal end for insertion in the anal canal of a patient and a proximal end for positioning exteriorly of the anal canal,
the cylindrically shaped side wall defining three lateral hemorrhoid-receiving apertures extending from the distal end toward the proximal end of the tubular body, and the lateral hemorrhoid-receiving apertures being spaced apart about the cylindrically shaped side wall to be positionable at the three o'clock, seven o'clock and eleven o'clock positions when inserted in the anal canal with the patient in the supine position.

13. The anoscope of claim 12 wherein the hemorrhoid-receiving apertures extend from the distal end of the tubular body to approximately half-way along the length of the tubular body.

14. The anoscope of claim 12, wherein the three hemorrhoid-receiving lateral apertures are each of equal circumference about the tubular body such that the sizes of the hemorrhoids of the patient can be visually graded by the observer by the degree of distension of the hemorrhoids from outside the cylinder through the hemorrhoid-receiving apertures of the anoscope.

15. The anoscope of claim 12, and further including a head mounted on the distal end of said tubular body that closes the distal end of the tubular body.

16. The anoscope of claim 12, and further including an obturator telescopically received in the proximal end of the tubular body of the anoscope, the obturator configured to rotate in the tubular body between positions for simultaneously closing or simultaneously opening the hemorrhoid-receiving apertures.

17. The anoscope of claim 16, and further including a head mounted on the distal end of the obturator that closes the distal end of the obturator.

18. The anoscope of claim 16, and further including an obturator telescopically received in the proximal end of the tubular body of the anoscope, the obturator configured to rotate in the tubular body between positions for opening the hemorrhoid-receiving apertures one at a time.

19. An anoscope for insertion in the anal canal of a human patient comprising:
an elongated tubular body having a distal end for insertion in the anal canal and a proximal end for positioning at the entrance of the anal canal,
the elongated tubular body defining three elongated hemorrhoid-receiving apertures located at the distal end at positions about the tubular body at the normal anatomic locations of the hemorrhoids in the anal canal for simultaneously presenting hemorrhoids of the anal canal of the patient in the interior of the tubular body without repositioning the anoscope and permitting simultaneous access to the hemorrhoids of the anal canal.

20. The anoscope of claim 19 and further including:
a bullet-shaped head mounted on the distal end of one of the tubular body and the obturator.

21. The anoscope of claim 19 and further including:
a handle mounted to the proximal end of the tubular body at a predetermined position with respect to the apertures such that by orienting the handle to a predetermined position with respect to the patient all of the hemorrhoid-receiving apertures are simultaneously oriented at the anatomical positions of the hemorrhoids of the patient.

22. The anoscope of claim 19, and further including:
an obturator telescopically received in the proximal end of the tubular body of the anoscope, the obturator configured to be rotated in the tubular body between positions for closing or opening at lease one of the hemorrhoid-receiving apertures.

23. The anoscope of claim 19, and further including an obturator telescopically received in the proximal end of the tubular body of the anoscope, the obturator configured to rotate in the tubular body between positions for opening the hemorrhoid-receiving apertures one at a time.

24. The anoscope of claim 19, and further including an obturator telescopically received in the proximal end of the tubular body of the anoscope, the obturator configured to rotate in the tubular body between positions for simultaneously opening all of the hemorrhoid-receiving apertures.

25. The anoscope of claim 19, and further including:
an obturator positioned within the tubular body of said anoscope, said obturator having a cylindrical side wall with elongated apertures formed there through that are sized and shaped to simultaneously align with the apertures of said anoscope,
such that the obturator can be rotated to have its apertures simultaneously register with the hemorrhoid-receiving apertures of the tubular body of the anoscope or rotated to simultaneously close the hemorrhoid-receiving apertures.

26. The anoscope of claim 25, wherein said obturator includes a handle that aligns with the handle of the anoscope when the apertures of the obturator are located at a predetermined position in the tubular body.

27. The anoscope of claim 19, and further including:
an obturator positioned within and rotatable with respect to the tubular body of said anoscope,
said obturator having a cylindrical side wall with a single elongated aperture formed there through that is sized and shaped to align with any one of the apertures of said anoscope,
alignment means for indicating the rotary position of the aperture of the obturator with respect to the tubular body,
such that the obturator can be rotated with respect to the tubular body to have its aperture register one at a time with selected ones of the hemorrhoid-receiving apertures of the tubular body or to close all of the hemorrhoid-receiving apertures of the tubular body.

28. The anoscope of claim 27, wherein the alignment means comprises a handle mounted to the obturator.

29. The anoscope of claim 28, wherein said handle of the tubular body is releasably connectable to said tubular body at more than one location about the tubular body.

30. The anoscope of claim 27, wherein the alignment means comprises a recess and a protrusion that registers with the recess.
